(19) United States
(12) Patent Application Publication
(10) Pub. No.: US 2023/0404077 A1
(43) Pub. Date: Dec. 21, 2023

(54) METHODS AND FORMULATIONS FOR CONTROLLING FLEA INFESTATIONS

(71) Applicant: EctoGuard, LLC, Carmel, IN (US)

(72) Inventors: Joseph R. WINKLE, Carmel, IN (US); Peter C. CANNING, Carmel, IN (US)

(21) Appl. No.: 17/841,572

(22) Filed: Jun. 15, 2022

(51) Int. Cl. A01N 43/22; A61P 33/14; A01N 45/02

(52) U.S. Cl. CPC A01N 43/22; A61P 33/14; A01N 45/02

(57) ABSTRACT

Methods and formulations for controlling a flea infestation. In some embodiments, the methods and formulations are suitable for direct application to an animal, such as an animal in need of treatment, or are suitable for direct application to textiles or other surfaces, and are configured to cause a lethal effect in all life cycle stages of fleas. A formulation includes at least one spinosyn, or a physiologically acceptable salt thereof, as an active ingredient. In some embodiments, the formulation also includes at least one carrier.

### CROSS-REFERENCE TO RELATED APPLICATIONS

N/A.

### GOVERNMENT RIGHTS STATEMENT

N/A.

### FIELD

The present technology is related generally to the control of ectoparasites such as companion animal fleas.

### BACKGROUND

The insects within the order Siphonaptera, commonly known as fleas, includes 2,500 species of small flightless insects that survive as external parasites of mammals and birds. Fleas live by consuming blood, or hematophagy, from their hosts. Fleas are problematic for humans and animals alike. For example, fleas serve as vectors for dog and cat bacterial diseases Rickettsia and Bartonella sp. Fleas also have the potential to transmit parasites such as tapeworms, as well as bacterial diseases such as murine typhus and cat scratch disease to humans. Additionally, flea allergic dermatitis associated with allergic responses of dogs to flea saliva causes significant discomfort (itching) in affected dogs.

Effective flea control includes treating the affected animal and the animal's environment or premises, which may include textiles such as blankets, bedding, and carpet, as well as other objects such as portable and stationary kennels which may be wood, plastic, and concrete. Elimination and/or control of one or more, and preferably all, life-cycle stages of the flea, is desired to prevent re-infestation of the animal and environment when ova (eggs) hatch and the larvae reach adulthood.

Flea infestations, and re-infestations, can occur at sites where animals are present, such as homes, garages, vehicles, kennels, hotels, motels, schools, hospitals, offices, veterinary clinics, and other commercial and industrial buildings. In order to combat such infestation or re-infestation, mechanical methods have been employed which include vacuuming carpets and upholstered furniture, or laundering washable textiles such as bedding in high heat. Integrated pest management practices used to eliminate fleas from premises inhabited by animals include the use of insecticides and pesticides. Sprays containing insecticides such as permethrin, imidacloprid, or dinotefuran are effective for killing adult fleas, but do not kill larvae or ova. Currently, the most effective premise treatments combine insecticides with insect growth regulators such as S-methoprene or pyriproxyfen which inhibit maturation of flea larvae and hatching of flea ova.

Oral administration of parasiticides for flea control, which may employ one or more active ingredients, has some advantages over topical administration. Oral administration eliminates the possibility of the active ingredient undergoing photodegradation, general degradation, washing off, or being dislodged. Spinosyns, naturally derived fermentation products, such as spinosad, may be used in oral delivery, and many commercial products demonstrate robust adult flea control. The commercial oral dose for spinosad in dogs for effective control as defined by regulatory authorities (≥90% mortality) is 30 to 60 mg/kg for dogs, and typically 45-90 mg/kg for cats. However, systemic exposure of spinosyns to dogs can result in negative side effects including emesis, depression/lethargy, anorexia, diarrhea and seizures. These side effects reduce the compliance of pet owners with the recommended dosing regimens for these orally administered products. One study shows that spinetoram, another spinosyn, is 5.5 times more active systemically than spinosad. Although this theoretically may cause fewer or less severe side effects as a result of lower dose requirements, spinetoram has not been commercialized for dogs.

Direct treatment of fleas on an animal, and in particular companion animals (such as dogs and cats) can be accomplished either by oral administration (systemic treatment) or topical application directly to the skin or fur of the animal. A variety of compounds such as fipronil, imidacloprid and selamectin can be useful in topical applications. However, all of these compounds tend to have substantially greater effectiveness against the adult parasites with some activity against immature stages, and poor to no control of ova via a true ovicidal action (inability to hatch).

### SUMMARY

Some embodiments advantageously provide methods and formulations for controlling a flea infestation, such as a flea infestation on an animal or a flea infestation on a textile or surface (premises surface).

In one embodiment, a method of controlling a flea infestation includes: producing a lethal effect in all life cycle stages of fleas on an animal by topically applying a formulation to an animal, the formulation including an effective amount of at least one spinosyn and a carrier.

In one aspect of the embodiment, the all life cycle stages of fleas on the animal includes ova, larvae, and adults.

In one aspect of the embodiment, the lethal effect in all life cycle stages of fleas on the animal is 100% mortality of flea ova, flea larvae, and adult fleas on the animal.

In one aspect of the embodiment, the animal is a companion animal.

In one aspect of the embodiment, the at least one spinosyn is spinosad.

In one aspect of the embodiment, the formulation is an organic liquid solution.

In one aspect of the embodiment, topically applying the formulation to the animal includes topically applying the formulation to a base of a skull of the animal.

In one aspect of the embodiment, topically applying the formulation to the animal includes topically applying the formulation in a dosage rate of between approximately 1 mg spinosyn per kg body weight of the animal and approximately 100 mg spinosyn per kg body weight of the animal.

In one aspect of the embodiment, topically applying the formulation to the animal includes topically applying the formulation in a dosage rate of between approximately 10 mg spinosyn per kg body weight of the animal and approximately 60 mg spinosyn per kg body weight of the animal.

In one aspect of the embodiment, the formulation is a liquid formulation; the at least one spinosyn is spinosad; and topically applying the formulation to the animal includes topically applying between approximately 0.5 mL to approximately 6 mL of the formulation to the animal.

In one embodiment, a method of controlling a flea infestation includes: producing a lethal effect in all life cycle stages of fleas on a premises surface by applying a formulation to a premises surface, the formulation including an effective amount of at least one spinosyn and a carrier.

In one aspect of the embodiment, the carrier is a non-organic carrier.

In one aspect of the embodiment, the lethal effect in all life cycle stages of fleas on the premises surface is 100% mortality of flea ova, flea larvae, and adult fleas on the premises surface.

In one aspect of the embodiment, applying the formulation to the premises surface includes at least one of spraying the formulation, soaking the premises surface in the formulation, and pouring the formulation onto the premises surface.

In one aspect of the embodiment, applying the formulation to the premises surface includes spraying the formulation onto the premises surface to evenly cover the premises surface.

In one aspect of the embodiment, the method further includes allowing the formulation to dry on the premises surface; and then removing the dried formulation from the premises surface.

In one aspect of the embodiment, the method further includes preparing the formulation before applying the formulation to the premises surface.

In one aspect of the embodiment, the effective amount of the at least one spinosyn is present in a wettable powder and the carrier is a non-organic carrier, the preparing the formulation before applying the formulation to the premises surface including mixing the wettable powder with the non-organic carrier.

In one aspect of the embodiment, the premises surface includes at least one of a textile, furniture upholstery, carpet, a rug, a pillow, bed linens, an animal bed, a towel, a plush toy, clothing, plastic, tile, wood, leather, concrete, glass, and metal.

In one embodiment, an anti-flea formulation includes: an effective amount of a spinosyn to cause a lethal effect in all life cycle stages of flea; and at least one carrier.

In one aspect of the embodiment, the at least one carrier is a non-organic carrier.

In one aspect of the embodiment, the spinosyn is present in an amount of between approximately 1000 ppm to approximately 4000 ppm.

In one aspect of the embodiment, the spinosyn is present in an amount of approximately 2500 ppm.

In one aspect of the embodiment, the formulation is one of an aqueous solution and an aqueous suspension.

In one aspect of the embodiment, the lethal effect in all life cycle stages of flea is a 100% mortality in flea ova, flea larvae, and adult fleas.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 is a flowchart of an exemplary method of controlling flea infestations on animals, such as companion animal fleas, in accordance with the present disclosure;

FIG. 2 is a flowchart of an exemplary method of controlling flea infestations on textiles or surfaces in the premises animals occupy, such as companion animal fleas, in accordance with the present disclosure; and

FIG. 3 is a flowchart of another exemplary method of controlling flea infestations on textiles or surfaces in the premises animals occupy, such as companion animal fleas, in accordance with the present disclosure.

### DETAILED DESCRIPTION

Before describing in detail exemplary embodiments, it is noted that the embodiments reside primarily in combinations of formulations and method steps related to controlling an infestation of parasitic insects, for example, Ctenocephcalides felis fleas, on a dog (or other animal, such as a companion animal) in need of treatment or on surfaces in the premises the animal occupies. In one embodiment, the present disclosure relates to a method of applying an anti-flea formulation topically to an animal to control a lice infestation, such as a companion animal lice infestation on a dog. In one embodiment, the present disclosure relates to a method of applying an anti-flea formulation to a textile or surface within the premises which an animal inhabits with a flea infestation or suspected flea infestation to control a companion animal flea infestation on premises surfaces. In one embodiment, the present disclosure relates to a formulation, such as an anti-flea formulation, containing at least one active ingredient sufficient to control an infestation of fleas at all life stages when applied topically to an affected animal. In one embodiment, the present disclosure relates to an anti-flea formulation containing at least one active ingredient sufficient to control an infestation of fleas at all life stages when applied directly to a textile or surface within the premises which the animal inhabits, rather than to an animal. In one embodiment, the fleas are companion animal fleas such as Ctenocephcalides felis. In one embodiment, as discussed below, the anti-flea formulation includes at least one spinosyn, or a physiologically acceptable derivative or salt thereof. Further, in one embodiment, the anti-flea formulation also includes an acceptable carrier.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

As used herein, the term “spinosyn” refers to an individual spinosyn factor (A, B, C, D, E, F, G, H, J, K, L, M, N, O, P, Q, R, S, T, U, V, W, or Y), a physiologically acceptable derivative of a spinosyn factor, such as an N-demethyl derivative of an individual spinosyn factor, a physiologically acceptable salt of a spinosyn factor, and/or any combination thereof. Further, the term “spinosyn” as used herein in the singular does not necessarily mean only one spinosyn factor and may include more than one spinosyn factor, as well as combinations of spinosyn factors and physiologically acceptable derivatives and/or physiologically acceptable salts thereof. Further, the term “spinosyns” as used herein refers to one or more spinosyn factor.

Spinosyns are naturally derived fermentation products. They are macrolides produced by cultivation of Saccharopolyspora spinosa, a species of bacteria. The fermentation produces multifactors, including spinosyn A and spinosyn D (also called A83543A and A83543D), which are the two spinosyns that are most active as insecticides. A product comprised mainly of these two spinosyns (approximately 85% A and 15% D) is available commercially under the name generic name spinosad. The name “spinosad” comes from a contraction of the spinosyns “A” and “D”. U.S. Pat. No. 6,063,771 describes spinosyns and is incorporated herein by reference in its entirety.

Each spinosyn factor has a 12-membered macrocyclic ring that is part of an unusual tetracyclic ring system to which two different sugars are attached: the amino-sugar forosamine and the neutral sugar 2N,3N,4N-tri-O-methylrhamnose. This unique structure sets spinosyns apart from other macrocyclic compounds.

Spinosyn A (A83543A) was the first spinosyn isolated and identified from the fermentation broth of Saccharopolyspora spinosa. Subsequent examination of the fermentation broth revealed that the parent strain of S. spinosa produced other spinosyns that have been labeled A to J (A83543A to J). Compared to spinosyn A, spinosyns B-J are characterized by differences in the substitution patterns on the amino group of the forosamine, at selected sites on the tetracyclic ring system and on 2N,3N,4N-tri-O-methylrhamnose. The strains of S. spinosa currently in use produce a mixture of spinosyns, of which the primary components are spinosyn A (about 85%) and spinosyn D (about 15%). Additional spinosyns, lettered from K to W, have been identified from mutant strains of S. spinosa.

U.S. Pat. Nos. 5,362,634, 5,496,932, and 5,571,901 describes spinosyns A-H and J (which are referred to therein as A83543 factors A, B, C, D, E, F, G, H and J), and salts thereof; U.S. Pat. No. 5,202,242 describes spinosyns L-N (which are referred to as A83543 factors L, M and N), their N-demethyl derivatives, and salts thereof; and U.S. Pat. Nos. 5,591,606 and 5,631,155 describe spinosyns Q-T (which are referred to therein as A83543 factors Q, R, S and T), their N-demethyl derivatives, and salts thereof. These patents are incorporated herein by reference in their entireties.

The spinosyns can react to form salts, and salts that are physiologically acceptable (referred to herein as “physiologically acceptable salts”) may also be used in the formulations and methods in accordance with the present disclosure, either alone or in combination with other ingredients, including other active ingredients. Further, as noted above, the term “spinosyn” as used herein includes physiologically acceptable salts of one or more spinosyns. Such salts are prepared using standard procedures for salt preparation. For example, spinosyn A can be neutralized with an appropriate acid to form an acid additional salt. In one embodiment, the formula includes a spinosyn that is or includes acid addition salts. Representative suitable acid addition salts include salts formed by reaction with either an organic or inorganic acid such as, for example, sulfuric, hydrochloric, phosphoric, acetic, succinic, citric, lactic, maleic, fumaric, cholic, pamoic, mucic, glutamic, camphoric, glutaric, glycolic, phthalic, tartaric, formic, lauric, stearic, salicylic, methanesulfonic, benzenesulfonic, sorbic, picric, benzoic, cinnamic and/or like acids.

In addition to the spinosyn, the methods and formulations of the present disclosure may optionally further include one or more other compounds that have activity against parasitic insects including fleas (for example, companion animal fleas), ticks, or lice including, but not limited to, other spinosyns, synthetic pyrethroids, natural pyrethins, organophosphates, organochlorines, carbamates, foramidines, avermectins, milbemycins, insect growth regulators (including chitin synthesis inhibitors, juvenile hormone analogs, and juvenile hormones), nitromethylenes, pyridines, and pyrazoles.

In one embodiment, a formulation (or anti-flea formulation, which term is used herein interchangeably with the term “formulation”) is a mixture or compound that is or contains an effective amount of active ingredient (for example, spinosyn in general, or spinosad in particular), either alone or in combination with one or more of the other types of compounds listed above, formulated into a product suitable for topical application to a dog or cat or to a textile or other surface within a premises, such as a premises the animal inhabits. In some embodiments, the formulation is a product that causes, or is capable of causing, 100% mortality (or effectively 100% mortality) to all flea life cycle stages, including ova, larvae, and adults. Further, in some embodiments the formulation is a product that causes, or is capable of causing mortality to all, or substantially all, fleas in all life cycle stages, including ova, larvae, and adults, on an animal or treated area of a premises surface. In some embodiments, the formulation causes a lethal effect in all life cycle stages of fleas.

In some embodiments, the formulation includes an effective amount (or ectoparasiticidal amount) of one or more active ingredients (for example, spinosyn) needed for a certain time (exposure time) at specified intervals (duration of activity) to control the parasitic insect infestation, such as a companion animal infestation.

In some embodiments, a carrier (or physiologically acceptable carrier, which term is used herein interchangeably with the term “carrier”) is any ingredient other than the active ingredient(s) in a formulation. In some embodiments, the choice of carrier(s) will depend on factors such as, for example, the particular anti-flea formulation being used, the active ingredient(s), the effect of the carrier on solubility and stability, the nature or form of the anti-flea formulation, the type of application method, and/or other considerations. The carrier(s) may include not only carrier materials that allow the active ingredient(s) to be distributed effectively, but also other materials such as adjuvants, binders, diluents, emulsifiers, and/or other inert or functional ingredients other than the active ingredient(s).

In some embodiments, the formulation is used to control a flea infestation, or the presence of at least one flea, such as C. felis, in one or more of the flea's life-cycle stages. In one embodiment, the flea infestation is a companion animal flea infestation, in which case the flea infestation may be referred to as a “companion animal flea infestation.” However, it will be understood that reference to companion animal fleas used herein may also include other species of fleas or other parasites, including companion animal ectoparasites. In one embodiment, the formulation is used to control a flea infestation by causing a lethal effect in all life cycle stages of fleas present on an animal and/or on or in a treated premises surface.

In some embodiments, controlling a flea infestation, such as a companion animal flea infestation, includes the prevention of parasitic insect infestations, such as companion animal flea infestations, and/or the treatment of existing parasitic insect infestations, such as companion animal flea infestations. In some embodiments, controlling a flea infestation includes preventing, minimizing, and/or eliminating an infestation by parasitic insects such as companion animal fleas on an animal and/or on premises surfaces. In some embodiments, the companion animal fleas are present in at least one life cycle stage selected from the group consisting of egg, larvae, and adult. In some embodiments, the companion animal fleas are present in at least one transitional life cycle stage between egg, larvae, and/or adult. Further, in some embodiments controlling a flea infestation includes controlling infestations and reinfestations, such as infestations and/or reinfestations of the animals themselves and textiles or other surfaces within the premises inhabited by the animal.

In some embodiments, the formulation is used on a premises surface, which may include any material or surface (including textiles) which is designed to come in contact with, or is capable of coming into contact with, animals and is capable of supporting a companion animal flea infestation in one or more of the flea's life-cycle stages. In some embodiments, a premises surface is a surface within the premises inhabited by a companion animal that has, is believed to have, or could have, a companion animal flea infestation therein or thereon. It will be understood that the term “premises surface” may also be used to refer to surfaces within a premises that has, or is believed to have, or could have, a companion animal flea infestation therein or thereon. Additionally, in some embodiments the premises surface includes textiles and/or other surfaces, such as porous, semi-porous, and nonporous surfaces or materials.

The companion animal flea infestation may be present on the animal. Typically, adult fleas inhabit the skin and hair of animals. The adults lay ova which can be attached to the animal's hair shafts or hair follicles. The ova are then shed or dislodged into the animal's environment within or on a textile, or other surface to which the companion animal flea ova may cling or be attached. Once the flea ova hatch, the larvae mature and the resulting adults re-infest the animal to complete their life-cycle. Comprehensive control of flea infestations requires elimination of the flea infestation on affected animals and surfaces in the premises the animal inhabits. As disclosed herein, companion animal flea infestations, such as flea infestations on dogs, can be treated by topically applying one embodiment of an anti-flea formulation to the skin of the affected animal. Further, another embodiment of the anti-flea formulation may be directly applied to textiles in the premises inhabited by the animal. The anti-flea formulation may also be applied to non-textile surfaces, if desired, such as porous and semi-porous surfaces (for example, wood, plaster, granite, stone, porous leather, and concrete) as well as nonporous surfaces, such as plastics, vinyl, glass, coated or waterproofed leather, tile, or the like.

Animals may be treated once per week, twice per month, once per month, or at other suitable intervals to achieve adequate exposure of all life-cycle stages of the companion animal fleas. It will also be understood that for premises applications, the active ingredient amounts and exposure times will vary depending upon a number of factors. For example, the duration of time for which the spinosyn is contact with companion animal fleas (exposure time) may be about 5 minutes to about 24 hours or more, about 30 minutes to about 12 hours, and about 60 minutes to about 6 hours. Also, in some embodiments, the surface to be treated is saturated with a formulation to assure contact with the companion animal fleas when a liquid formulation is used. However, it will be understood that the methods and formulations will be effective in amounts which do not saturate the surface.

Thus, in some embodiments, the methods and formulations in accordance with the present disclosure are suitable for direct application to an animal in need of treatment and are configured to cause a lethal effect in all life cycle stages of fleas. In some embodiments, the methods and formulations in accordance with the present disclosure are suitable for direct application to textiles or other surfaces within a premises, which have, or are believed to have, a companion animal flea infestation therein or thereon, and are configured to cause a lethal effect in all life cycle stages of fleas. Whether applied to an animal in need of treatment or to premises surfaces, the formulations contain spinosyn (for example, spinosad) as an active ingredient. In some embodiments, the formulation also includes one or more physiologically acceptable carriers.

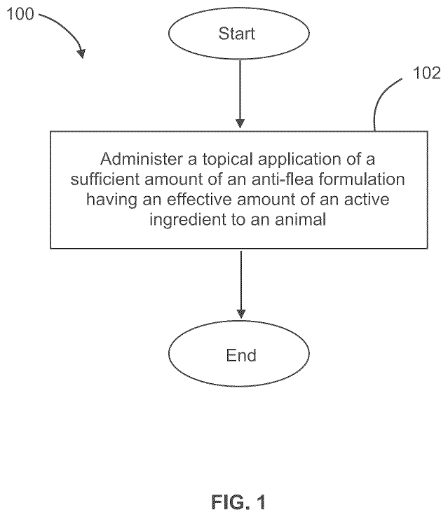

Referring now to FIG. 1, a flowchart of an exemplary method 100 of controlling a flea infestation, such as a companion animal flea infestation, is shown. As discussed above, in one embodiment, the method 100 generally includes a step 102 of administering a topical application of a formulation including an effective amount of an active ingredient, such as at least one spinosyn, to an animal in need of treatment. For example, the animal that is in need of treatment may have a companion animal flea infestation, or may be believed to have a companion animal flea infestation. In one embodiment, a formulation for use in the method of FIG. 1 is suitable for topical application to a companion animal, such as a dog, that has, or is believed to have, a companion animal flea infestation, without causing injury or passing through the skin barrier to become systemic in the animal. Although a single step is shown in the method of FIG. 1, it will be understood that additional steps may be included, such as pre-mixing or preparing the formulation before use.

Continuing to refer to FIG. 1, in some embodiments the formulation includes spinosyn as the active ingredient and is formulated in solution or suspension for topical application onto animals. Such formulations include, but are not limited to, solutions, gels, suspensions, pastes, lotions, foams, sprays, and powders (such as wettable powders) containing the active ingredient (for example, spinosyn in general, or spinosad in particular). The formulation contains an effective amount of a spinosyn, either alone or in combination with one or more carriers or other active ingredients. In some embodiments, carriers include, but are not limited to, excipients, diluents, stabilizers, water, and/or adjuvants. Additionally, in some embodiments the formulation is provided or sold as a ready-to-use formulation that does not require further mixing or preparation (other than, in some circumstances, shaking or agitation before use to re-suspend any solid particles that have separated out of solution). In other embodiments, the formulation may be sold as one or more components that are to be mixed, blended, diluted, or otherwise require some preparation before use. In some embodiments, the formulation is a wettable powder that the user mixes into or adds to water or other carrier in an application vessel or other container to create a diluted aqueous suspension and/or diluted aqueous solution. For example, the concentrate formulation, wettable powder, or other component may be sold in a bottle, jar, in individual pre-measured dosage vials, single-use quick-dissolve packets or sachets, or in other suitable containers or packaging devices, and which may or may not include additional room for adding a suitable carrier.

In some embodiments, the formulation is an organic solution of a spinosyn, such as spinosad, that includes one or more organic solvents that do not allow penetration of the spinosyn into the vascular system, such that the spinosyn is essentially surface-oriented on the animal's body and not systemically moving in the blood. In some embodiments, the one or more solvents allow the spinosyn to spread through the animal's sebaceous glands, but do not allow the spinosyn to pass through the animal's skin. In some embodiments, the formulation includes one or more solvents including, but not limited to, polyethylene glycol (PEG) solvents, organic alcohols and/or oils. In other words, the formulation is substantially predominantly non-systemic, and the formulation (and active ingredient) may be in contact with the ova on the animal's skin and near, or on, hair follicles. In some embodiments, the solvents include, but are not limited to, certain glycol ethers, aromatic or sugar alcohols, vitamin E derivatives, propylene carbonate, certain polyethylene glycols, and/or additional carriers that are known to prevent, or not promote, movement of the formulation (and, in particular, the active ingredient) across the skin barrier.

For animal use, topical formulations are typically dosed by applying a volume of formulation with a set amount of ectoparasiticide (active ingredient) to a given weight range of the animal. Oral formulations also typically include a set amount of ectoparasiticide, but dose is generally expressed as milligrams (mg) ectoparasiticide per kilogram (kg) animal weight, or mg/kg. Dog weight ranges are typically the same between oral and topical routes of delivery, with common breakdowns being 2-5 kg, 5-10 kg, 10-20 kg, 20-40 kg, and above 40 kg. The volume of applied topical formulations increases as the weight ranges increase, and the size of an oral tablet or chew increases as the same weight ranges increase. Minimum effective dose (MED), or that does necessary for a dog to receive 90% or greater flea control from a given oral or topical delivery method, also is normally expressed as mg of ectoparasiticide per kg of dog body weight. MED multiplied by the highest weight in a weight range determines the amount of ectoparasiticide given in that weight range. For example, an MED for an ectoparasiticide might be 10 mg/kg. For a dog weighing 15 kg, which falls in the 10-20 kg range, 200 mg of an ectoparasiticide (MED of 10 mg/kg multiplied by 20 kg as upper limit in that weight range) would be given in the solid oral dosage form to that dog. If the same ectoparasiticide had topical activity as well, one might expect that the dog would receive the same 200 mg in that dose band. A 10% by weight solution formulation might require 2 mL for dogs in the 10-20 kg weight band, and volumes of formulations are chosen to deliver the ectoparasiticide ingredient without causing run-off of the topical formula. At the same innate activity of 10 mg/kg, 2 mL of a topical formulation would deliver 200 mg, just as the oral formulation does. Thus, one can extrapolate oral and topical doses based on innate activity of the ectoparasiticide ingredient.

In rare cases, an ectoparasiticide class and individual ectoparasiticide within that class may exhibit both oral and topical activity against ectoparasiticides, especially fleas. For example, spinetoram is more innately effective than some other types of spinosyns (in the case of spinosad, for example, spinetoram is about 3 times to about 5.5 times more innately effective when administered orally). Spinetoram also is known to be active topically in cats at a corresponding extrapolated value of around 10 mg/kg. Therefore, in looking at spinosad, one would expect that a topical formulation for dogs might require closer to 40 to 55 mg/kg as an extrapolated dose/MED due to the greater innate activity of spinetoram as compared to spinosad. As noted above, there is a practical limitation (approximately 0.4 to approximately 6.0 mL) as to how much volume that can be applied to a dog without the formulation running off the animal, adhering to fabrics/furniture as the animal comes into contact with those materials, and/or concerns over residue transfer to humans with excessive volumes applied. It also may be difficult to achieve solubility of these ectoparasite compounds in the limited number of solvents available. For dogs in the 20-40 kg body weight range, one would need 55 mg/kg multiplied by kg, or 2200 mg (or 2.2 g of spinosad) to be delivered to a dog in that range in about 5 mL of solution. This would approximately translate to about a 40-50% by weight formulation of just active ingredient (ectoparasiticide) itself, which would also be likely to cause viscous clumping of the hair, hair loss, potential itching, and/or unwelcomed damage to household or premises materials and/or furnishings due to the organic solvents typically used in spot-on formulations. However, it has been surprisingly found that approximately the same volume of a topical formulation containing spinosad as would be expected for use with a topical formulation containing spinetoram produces a lethal effect (for example, 95-100% mortality or effectively 100% mortality) in all life cycle stages of fleas when applied topically, despite being less innately active than spinetoram.

Continuing to refer to FIG. 1, in one embodiment a spinosyn, for example, spinosad, may be administered to an animal in need of treatment by any suitable route, such as via spray or direct spot-on treatment. In one exemplary method of application, an anti-flea formulation containing spinosyn (for example, spinosad) as an active ingredient is applied to the base of the animal's skull in a volume of approximately 0.5 mL to approximately 6 mL (±0.2 mL) of the formulation, based on the bodyweight of the animal. However, it will be understood that in some embodiments the formulation is applied to other locations, instead of or in addition to at the base of the skull, such as along the animal's back or at the midline of the back. In some exemplary methods of application, dose ranges for a spinosyn, for example, spinosad, in the formulation are from approximately 0.1 mg/kg to approximately 100 mg/kg (±0.05 mg/kg) of body weight of the animal. In some exemplary methods of application, dose ranges for a spinosyn, for example, spinosad, in the therapeutic on-animal uses and formulations range from approximately 1 mg/kg to approximately 75 mg/kg (±0.5 mg/kg) of body weight of the animal. In some exemplary methods of application, dose ranges for a spinosyn, for example, spinosad, in the therapeutic on-animal uses and formulations range from approximately 10 mg/kg to approximately 60 mg/kg (±2 mg/kg) of body weight of the animal. Thus, in one exemplary method of application, the volume of formulation applied to the animal is sufficient to supply spinosyn in such dose ranges.

Continuing to refer to FIG. 1, in one embodiment the formulations in accordance with the present disclosure are suitable for direct application to a companion animal, such as a dog. In some embodiments, the formulation is an organic solution containing a spinosyn (such as spinosad) in a concentration of between approximately 0.1% to approximately 50% (±0.05%) by weight of the formulation. In other embodiments, the formulation is an organic solution containing a spinosyn (such as spinosad) in a concentration of between approximately 1% to approximately 30% (±0.5%) by weight of the formulation. In other embodiments, the formulation is an organic solution containing a spinosyn (such as spinosad) in a concentration of between approximately 5% and approximately 20% (±1%) by weight of the formulation.

Thus, in some embodiments (for example, as shown in FIG. 1), the anti-flea formulation is applied directly to the animal in need of treatment. Further, in one embodiment the formulation is applied directly to the animal in need of treatment in one step, such as when the formulation is a ready-to-use liquid, foam, spray, or the like. For example, a user may apply the formulation to the animal using one or more spray bottles or spot-on treatment applicators (for example, ampules, droppers, daubers, or the like). In one embodiment, a sufficient amount of the anti-flea formulation is applied to the animal. The amount that is a sufficient amount may depend on the form of the formulation, the carrier(s) used, the size and/or weight of the animal to be treated, the active ingredient used, and/or other factors. In one embodiment, the formulation is applied topically (on the animal's skin/hair), such as at the base of the skull, between the shoulder blades, or at another suitable location. The formulation causes, or is capable of causing, 100% mortality (or effectively 100% mortality) of all flea life cycle stages, including ova, larvae, and adults, and a sufficient amount of the formula is an amount that provides an effective amount of active ingredient to cause 100% (or effectively 100% mortality) mortality of all flea life cycle stages on the animal. In some embodiments, topical application of the formulation to the animal has a lethal effect on all flea life cycle stages on the animal. In some embodiments, the lethal effect is the death of all, or substantially all, fleas in all life cycle stages on the animal. Additionally, in some embodiments the formulation is sold to a retailer or end user as a ready-to-use formulation that can be applied to an animal without further preparation (other than, in some circumstances, shaking or agitation to re-suspend any solid particles that have separated out of solution, or the like).

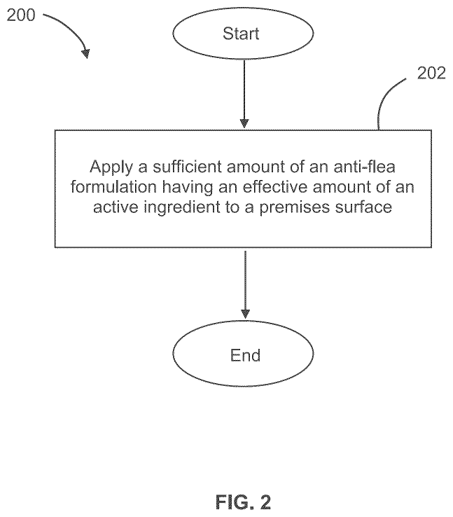

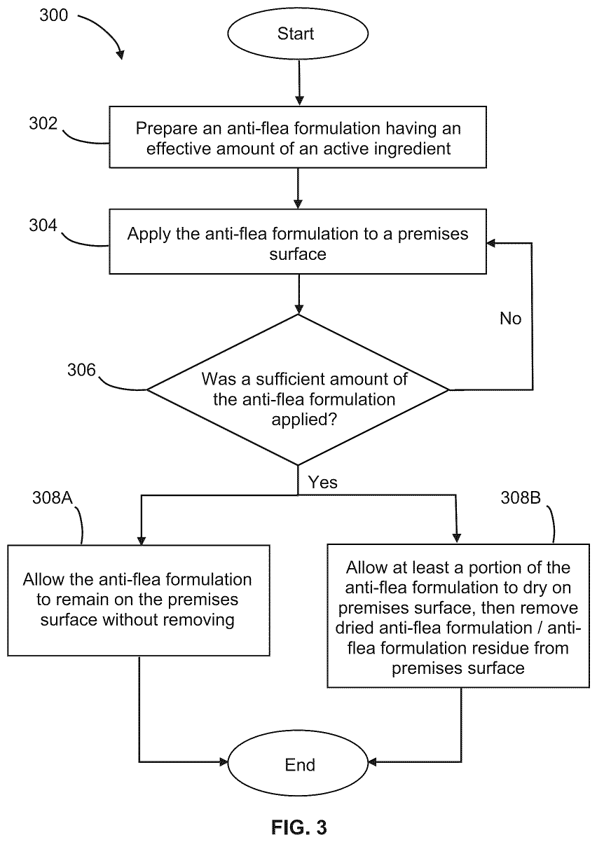

Referring now to FIGS. 2 and 3, methods of applying a formulation (for example, an anti-flea formulation) to a textile or surface in need of treatment are shown (which may also be referred to herein as “premises surfaces”). FIG. 2 shows a single-step method in which a pre-mixed or prepared formulation is applied to a premises surface, and FIG. 3 shows a multi-step method in which a formulation is first mixed or prepared and then applied to a premises surface.

Continuing to refer to FIGS. 2 and 3, in some embodiments the textile or surface that is in need of treatment may have a companion animal flea infestation, or may be believed to have a companion animal flea infestation. In one embodiment, the formulation includes an effective amount of spinosyn (for example, spinosad) as the active ingredient. As discussed below regarding Tables 1-3, the formulations in accordance with the present disclosure are effective in controlling all life stages of a companion animal flea infestation, including eggs, larvae, and adult fleas, when applied to a premises surface, such as a textile or other surface including porous, semi-porous, and nonporous surfaces. This is in contrast to currently known formulations for and methods of treating a textile or surface having a companion animal flea infestation by heat treatment, vacuum treatment, and/or other methods that do not include applying an anti-flea formulation including at least one spinosyn to the textile or surface.

Continuing to refer to FIGS. 2 and 3, surfaces suitable for treatment with one or more anti-flea formulations in accordance with the present disclosure include textiles composed of natural or synthetic fibers, as well as textile blends in woven or non-woven form, such as knit goods, yarns or fibers, and which come in contact with animals (or are capable of coming into contact with animals) and which contain or are capable of containing one or more companion animal fleas at one or more life-cycle stages. Natural fibers include, for example, cotton, wool, silk, jute, and hemp. Synthetic fibers include, for example, polyamides, polyesters, polyacryl nitrites, polyolefins (for example, polypropylene or polyethylene), TEFLON®, and/or mixtures of fibers, for example mixtures of synthetic and natural fibers. Textiles may be in form of coverings, for example blankets, pillows, bed linens, duvets, cushions and dog (or pet) beds, curtains, upholstery fabrics, car seats, carpeting, rugs, plush toys and stuffed animals, towels, and/or the like. Other suitable surfaces include, but are not limited to, porous, semi-porous, and nonporous surfaces such as wood, plastic, glass, and metal. Additionally, it will be understood that the formulations of the present disclosure may be applied to textiles and/or surfaces other than those explicitly disclosed herein.

Continuing to refer to FIGS. 2 and 3, regardless of the method of application of the formulation, in one embodiment the formulation includes an active amount of spinosyn. In one embodiment, the formulation includes approximately 2500 ppm (or 0.25% by weight) spinosad. However, it will be understood that other effective amounts of spinosyn, and spinosyns other than spinosad and/or physiologically acceptable spinosyn salt(s), may be used, such as between approximately 50 ppm (or 0.005% by weight) and approximately 5000 ppm (or 0.5% by weight) spinosyn (±10 ppm or 0.001% by weight), such as spinosad, including between approximately 1000 ppm (or 0.0.1% by weight) and approximately 4000 ppm (or 0.4% by weight) spinosyn, such as spinosad.

Continuing to refer to FIGS. 2 and 3, suitable formulations include, but are not limited to, liquids, gels, pastes, lotions, foams, sprays, and/or powders containing the active ingredient(s) (for example, spinosyn in general, or spinosad in particular). In some embodiments, such formulations include one or more physiologically acceptable carriers. In some embodiments, the physiologically acceptable carriers include, but are not limited to, excipients, diluents, stabilizers, water, adjuvants, and/or other suitable materials. In some embodiments, the one or more physiologically acceptable carriers includes a non-organic or inorganic carrier

Referring now to FIG. 2, a flowchart of a first exemplary method 200 of controlling a flea infestation, such as a companion animal flea infestation, on a textile or surface in need of treatment is shown. In one embodiment, the method 200 generally includes a step 202 of applying a formulation (for example, an anti-flea formulation) including an effective amount of at least one spinosyn to a premises surface.

Continuing to refer to FIG. 2, in some embodiments the formulation for use on premises surfaces include wettable powder formulations having from approximately 1% to approximately 80% active ingredient (for example, spinosyn or spinosad) by weight. In some embodiments, the spinosyn active ingredient is formulated as an aqueous suspension or solution, or as a dilute aqueous solution, and is premixed and sold to the end user or retailer as a ready-to-use formulation. In one embodiment, the spinosyn is present in an anti-flea formulation in an amount from approximately 50 ppm (or 0.005% by weight) to approximately 10000 ppm (or 1.0% by weight) (±10 ppm or 0.001% by weight). In one embodiment, the spinosyn(s) is present in an amount from approximately 500 ppm (or 0.050% by weight) to approximately 5000 ppm (or 0.5% by weight) (±100 ppm or 0.010% by weight). In one embodiment, the spinosyn(s) is present in an amount from approximately 1000 ppm (or 0.1% by weight) to approximately 4000 ppm (0.4% by weight) (±100 ppm or 0.010% by weight). In one embodiment, the spinosyn(s) is present in an amount from approximately 2000 ppm (or 0.2% by weight) to approximately 3000 ppm (0.3% by weight) (±100 ppm or 0.010% by weight). In one embodiment, the formulation includes approximately 2500 ppm (or 0.25% by weight) spinosyn.

Continuing to refer to FIG. 2, in some embodiments the formulation is sold as a ready-to-use formulation. However, in some embodiments the formulation is prepared by the manufacturer/producer, before sale to the end user, as a pre-diluted, or concentrate, formulation that is then diluted by the same or different manufacturer/producer to achieve the ready-to-use formulation that is offered for sale to the end user. In some embodiments, a concentrate formulation is from approximately 0.5% to approximately 45%, including 44.2%, by weight of active ingredient (±0.1%). In one embodiment, the spinosyn is completely or partially soluble based on the concentration in water having a pH of 6.5 to 7. In some embodiments, the formulation includes organic acids such as citric acid or other agents as antioxidants to increase the spinosyn stability. In some embodiments, the formulation includes primarily non-ionic surfactants at levels of 0.05% to 0.1% by weight at the time of use, to aid in solubilizing a spinosyn in an aqueous environment. In some embodiments, the formulation includes nonionic surfactants, such as primary and secondary alcohol ethoxylates, including, but not limited to, ethoxylated surfactants such as C₈-C₂₀ aliphatic alcohols ethoxylated with an average of from 1-20 moles of ethylene oxide per mole of alcohol and/or C₁₀-C₁₅ primary and secondary aliphatic alcohols ethoxylated with an average of from 1-10 moles of ethylene oxide per mole of alcohol, and/or non-ethoxylated nonionic surfactants such as alkylpolyglycosides, glycerol monoethers, and/or polyhydroxyamides (glucamide). In some embodiments, the formulation includes water-miscible vehicles, including but not limited to ethanol, 1,3-butanediol, liquid polyethylene glycol (for example, polyethylene glycol 300 and/or polyethylene glycol 400), propylene glycol, glycerin, N-methyl-2-pyrrolidone, N,N-dimethylacetamide, dimethyl sulfoxide, benzyl alcohol, and/or isopropanol. In some embodiments, the formulation includes other carriers such as: anionic surfactants; xanthan gum, gum Arabic, and/or any other suitable viscosity builder; inorganic salts such as magnesium or aluminum carbonates; glycol as a freeze point depression agent; an antimicrobial agent; an ionic or neutral dispersant; and/or other suitable materials.

Thus, in some embodiments (for example, as shown in FIG. 2), the anti-flea formulation is applied to the textile or surface in one step, such as when the anti-flea formulation is a ready-to-use liquid, foam, spray, or the like. For example, a user may apply the formulation to the premises surface using one or more spray bottles, or may pour the formulation over the premises surface. In one embodiment, a sufficient amount of the anti-flea formulation is applied to the premises surface, such as a textile or other surface, in a single step without the need for dilution or other pre-application preparation steps. The amount that is a sufficient amount may depend on the form of the formulation, the carrier(s) used, the type of textile or surface, and/or other factors. In one embodiment, an entirety of a predetermined area of the textile or surface (for example, an animal-contacting surface of a dog bed) is coated or saturated with the formulation, or is otherwise exposed to an amount of the anti-flea formulation. The formulation causes, or is capable of causing, 100% mortality (or effectively 100% mortality) of all flea life cycle stages, including ova, larvae, and adults, and a sufficient amount of the formula is an amount that provides an effective amount of active ingredient to cause 100% mortality (or effectively 100% mortality) of all flea life cycle stages on the premises surface. In some embodiments, topical application of the formulation to the premises surface has a lethal effect on all flea life cycle stages on the premises surface. In some embodiments, the lethal effect is the death of all, or substantially all, fleas in all life cycle stages on the premises surface (or at least to the area(s) of the premises surface to which the formulation is applied). Additionally, in some embodiments the formulation is sold to a retailer or end user as a ready-to-use formulation that can be applied to a premises surface without further preparation (other than, in some circumstances, shaking or agitation to re-suspend any solid particles that have separated out of solution, or the like).

Referring now to FIG. 3, a flowchart of another exemplary method 300 of controlling a flea infestation, such as a companion animal flea infestation, is shown. In one embodiment, the anti-flea formulation includes an active amount of spinosyn, such as approximately 2500 ppm (or 0.25% by weight) spinosad at the time of use. However, it will be understood that other effective amounts of spinosyn may be used, such as between approximately 50 ppm (or 0.005% by weight) and approximately 5000 ppm (or 0.5% by weight) spinosyn (±10 ppm or 0.001% by weight) at the time of use, including between approximately 1000 ppm (or 0.1% by weight) and approximately 4000 ppm (or 0.4% by weight) spinosyn at the time of use. In one embodiment, the anti-flea formulation includes an amount of spinosyn that is diluted or mixed with one or more carriers or other active ingredients such that the anti-flea formulation includes, for example, 2500 ppm (or 0.25% by weight) spinosad in the prepared formulation at the time of use.

Continuing to refer to FIG. 3, in a first step 302 of the method 300, a user prepares an anti-flea formulation before applying it to the premises surface. In one embodiment, the user prepares the formulation by mixing a wettable powder containing an effective amount of spinosyn with a physiologically acceptable carrier and placing the formulation into a suitable application vessel, such as a spray bottle, squirt bottle, jar, bowl, dauber or blotter, or the like. In one embodiment, the user prepares the formulation by diluting a liquid concentrate formulation containing an effective amount of spinosyn with a physiologically acceptable carrier (for example, water) and placing the formulation into a suitable application vessel, such as a spray bottle, squirt bottle, jar, bowl, dauber or blotter, or the like. In another embodiment, the user prepares the formulation by mixing an aqueous suspension of an effective amount of spinosyn and placing the anti-flea formulation into a suitable application vessel, such as a spray bottle, squirt bottle, jar, bowl, dauber or blotter, or the like. However, it will be understood that other methods of preparing a formulation for use may be used, such as diluting, wetting, mixing, blending, stirring, or other pre-application preparation steps.

Continuing to refer to FIG. 3, in a second step 304, the user applies the formulation to a premises surface from the application vessel. In an optional third step 306, the user determines whether a sufficient amount of the formulation has been applied to the premises surface. In one non-limiting example, a sufficient amount of formulation is a light mist that entirely covers a desired surface area or area of treatment. In another non-limiting example, a sufficient amount of formulation is the amount required to completely saturate a textile or surface. If the user determines that a sufficient amount of the formulation has not been applied to the textile or surface, the user then makes one or more additional applications (that is, applies at least one additional amount of the formulation to the textile or surface). In one optional fourth step 308A, the user allows the formulation on the premises surface to dry, and the dried formulation or formulation residue is not removed from the premises surface, before use thereof and/or before animal or human contact with the premises surface. In an alternative optional fourth 308B, the user allows at least a portion of the formulation on the premises surface to dry, and then the user removes at least a portion of the dried formulation and/or formulation residue from the premises surface before use thereof and/or before animal or human contact with the premises surface.

Thus, in some embodiments (for example, as shown in FIG. 3), the anti-flea formulation is an amount of an active ingredient that is pre-mixed or prepared with a suitable liquid carrier before the formulation is applied onto the textile or surface in need of treatment. In one embodiment, the formulation is a wettable powder of spinosyn that is pre-mixed or prepared with a suitable liquid carrier before the formulation is applied onto a premises surface. For example, a user may first prepare the formulation and then apply the formulation to the premises surface using one or more spray bottles, or may be poured over the premises surface. In one embodiment, a sufficient amount of the anti-flea formulation is applied to the premises surface, such as a textile or other surface. The amount that is a sufficient amount may depend on the form of the formulation, the carrier(s) used, the type of textile or surface, and/or other factors. In one embodiment, an entirety of a predetermined area of the textile or surface (for example, an animal-contacting surface of a dog bed) is coated or saturated with the formulation, or is otherwise exposed to an amount of the anti-flea formulation. The formulation causes, or is capable of causing, 100% mortality (or effectively 100% mortality) of all flea life cycle stages, including ova, larvae, and adults, and a sufficient amount of the formula is an amount that provides an effective amount of active ingredient to cause 100% mortality (or effectively 100% mortality) of all flea life cycle stages on the animal. In some embodiments, topical application of the formulation to the premises surface has a lethal effect on all flea life cycle stages on the premises surface. In some embodiments, the lethal effect is the death of all, or substantially all, fleas in all life cycle stages on the premises surface (or at least to the area(s) of the premises surface to which the formulation is applied). In some embodiments, the formulation may be sold as one or more components that are to be mixed, blended, diluted, or otherwise require some preparation before use. In some embodiments, the formulation is a wettable powder that the user mixes into or adds to water or other carrier in an application vessel or other container to create a diluted aqueous suspension and/or diluted aqueous solution. For example, the concentrate formulation, wettable powder, or other component may be sold in a bottle, jar, in individual pre-measured dosage vials, single-use quick-dissolve packets or sachets, or in other suitable containers or packaging devices, and which may or may not include additional room for adding a suitable carrier.

### Example 1

In one exemplary first study (Study 1), the impact of direct application of an anti-flea formulation according to the present disclosure on flea viability in all life cycle stages was evaluated. In this exemplary Study 1, CONSERVE® NATURALYTE® Insect Control (0.5% spinosad) (Dow Agrosciences LLC, Indianapolis, IN), a product that is labeled for use in home gardens, lawns and ornamentals, and fruit trees, was diluted with water to achieve a spinosad concentration of 2500 ppm (0.25% on a weight basis).

Cat fleas (Ctenocephalides felis) were used in this exemplary Study 1, and were maintained at the testing laboratory used for all mortality and ovicidal assays. All life-cycle stages of the C. felis were active/viable prior and up to infestation and were healthy in appearance at the start of each evaluation. Ten (10) individuals from each life-cycle stage of C. felis (egg, larva, and adult) were collected from rearing containers and were placed in standard laboratory knockdown containers (for adult fleas, 12-oz. plastic cups lined with a coffee filter were used) or filter-paper-lined Petri dishes (for flea larvae and eggs) for treatment. Untreated controls were used for comparison. A trigger-spray bottle with droplets of greater than 50μ (microns) was used for each formulation application. Each spray bottle was weighed before and after each replicate to determine the approximate amount of formulation applied to each set of seven (7) replicates in the treated groups. The spinosad suspension concentrate formulation was shaken well and sprayed directly on the selected life-cycle stage of C. felis for a treatment application rate of approximately 0.56-0.67 g formula/replicate (1.4-1.7 mg of spinosad) per test container. The adult and larval C. felis were then transferred to clean test containers one hour after treatment, where they remained for the duration of the evaluation. Eggs were both treated and allowed to remain in the plastic Petri dishes lined with filter paper (and were not transferred to clean test containers).

During scoring, adult C. felis from both the treatment group and the control group were stimulated by the evaluator's breath and/or by body warmth provided by the user's holding the containment vessel. This was intended to incite any movement or host-seeking activity of the adult flea. Larvae were evaluated with the aid of a dissecting microscope to determine movement. Eggs were scored for hatch with the aid of a dissecting microscope. Mortality in both the treatment group and the control group was assessed to determine efficacy of the experimental spinosad formulation: at 30 seconds, 30 minutes, one hour, two hours, four hours, 24 hours, and 48 hours for adults; at one hour, two hours, four hours, 24 hours, and 48 hours for larvae; and at one day and two days for eggs. Comparisons of means were evaluated and the percent effect was pooled for each test group. Data was collected for each replicate, but replicates were pooled to determine a mean percent effect for that treatment group.

The suspension concentrate formulation was effective in killing adult, larvae, and egg stages of C. felis when applied as a direct application treatment when compared with the control group. As shown below in Table 1, direct application of the formulation (average 1.4 mg of active ingredient (AI) applied) to adult C. felis demonstrated moderate mortality rates (31.3% to 67.5%) through the four-hour time point after treatment with the spinosad formulation. However, by the 24-hour time point after treatment, mortality increased to 90% with the remaining 10% being moribund (that is, in a dying state or about to die), such that the overall mortality for adults was effectively at 100%. The untreated control adult flea mortality remained low (less than 3%).

TABLE 1
Table 1. Comparison of effects of treatment of adult
fleas with spinosyn formulation to untreated control.

| Adults | Avg. % mortality (n = 7) | | | | | | Avg. amount AI |
|---|---|---|---|---|---|---|---|
| Test Group | 30 sec. | 30 min. | 1 hour | 2 hours | 4 hours | 24 hours | applied (mg) |
| Spinosad | 31.3 | 38.8 | 45.0 | 61.3 | 67.5 | 90.0 | 1.4 |
| suspension concentrate | | | | | | | |
| (spinosyn formulation) | | | | | | | |
| Untreated control | 2.9 | 2.9 | 2.9 | 1.4 | 0.0 | 2.9 | — |

Further, as is shown below in Table 2, direct application of the formulation (average 1.5 mg of active ingredient (AI) applied) to C. felis larvae was very effective, with 100% mortality from the one-hour timepoint after treatment through the 48-hour time point after treatment, with no recovery observed. In contrast, the untreated control mortality of larvae was less than 13%, which is not unexpected in this type of bioassays.

TABLE 2
Table 2. Comparison of effects of treatment of flea larvae
with spinosyn formulation to untreated control.

| Larvae | Avg. % mortality (n = 7) | | | | | Avg. amount AI |
|---|---|---|---|---|---|---|
| Test Group | 1 hour | 2 hours | 5 hours | 24 hours | 48 hours | applied (mg) |
| Spinosad | 100 | 100 | 100 | 100 | 100 | 1.5 |
| suspension concentrate | | | | | | |
| (spinosyn formulation) | | | | | | |
| Untreated control | 0.0 | 1.4 | 2.9 | 8.6 | 12.9 | — |

Still further, as shown below in Table 3, the ovicidal activity of the spinosad formulation (average 1.7 mg of active ingredient (AI) applied) against C. felis eggs was excellent, demonstrating 100% inhibition of egg hatch (that is, 0% hatch or 100% mortality) as a direct treatment application with the spinosyn formulation. Eggs hatch in the untreated control after two days was 32.9%, but would have been higher after three days post-treatment since peak egg hatch typically occurs three days after oviposition.

TABLE 3
Comparison of effects of treatment of flea eggs with
spinosyn formulation to untreated control.

| Eggs | Avg. % hatch (n = 7) | | Avg. amount AI |
|---|---|---|---|
| Test Group | 1 day | 2 days | applied (mg) |
| Spinosad suspension | 0.0 | 0.0 | 1.7 |
| concentrate | | | |
| (spinosyn formulation) | | | |
| Untreated control | 1.4 | 32.9 | — |

Thus, the exemplary spinosyn formulation containing 2500 ppm of spinosad was effective in killing adult, larvae, and egg life cycle stages of C. felis when applied as a direct application treatment demonstrating efficacy (that is, producing mortality of) between 90% and 100%. The observed levels of mortality for adults and larvae were consistent with expected efficacy rates required for regulatory approval for premises products. The 100% inhibition of flea egg hatchability following direct exposure is unprecedented and has not been documented previously for spinosad.

### Example 2

An exemplary second study (Study 2) was performed to evaluate the colorfastness and integrity of commonly used textiles following exposure to an anti-flea formulation having a concentration of 2500 ppm Spinosad.

In the exemplary Study 2, an anti-flea formulation having 2500 ppm spinosad (for example, CONSERVE® NATURALYTE® Insect Control (spinosad 0.5%)) was used. A spinosad concentrate having 5000 ppm was diluted with water to achieve a formulation having a concentration of 2500 ppm spinosad, and the diluted formulation was then mixed by agitation to ensure a homogeneous suspension immediately prior to application to fabric swatches.

In the exemplary Study 2, fabric swatches measuring approximately 6 inches square were used. The fabric samples included cotton, polyester, nylon, and olefin in a variety of finishes including percale, linen, fleece, and woven upholstery. Further, the fabric swatches included different colors representing a range of dyes commonly used in commercial fabrics. The fabric swatches were placed onto a water-absorbent barrier in a grid pattern. Swatches were adequately separated to ensure no exposure to the active ingredient from adjacent swatches occurred during application. A rigid barrier was held vertically against the midline of each fabric sample to limit the area of exposure to the active ingredient to one half of each fabric swatch, which enabled each sample to serve as its own untreated control (that is, each fabric swatch as divided into a treated portion and an untreated portion). The right lower corner of each sample was clipped or otherwise marked to indicate the treated side of the sample.

Two sprays, approximately 2.5 ml, of the anti-flea formulation were sprayed onto half of each fabric swatch using a single spray bottle to ensure even saturation of the treated portion of each of the fabric swatches. The fabric swatches were then left in place to allow the anti-flea formulation (and, therefore, the active ingredient) to dry on the fabric for approximately two hours. The dried fabric swatches were then washed in a residential front load washing machine using cold water on the gentle cycle and a detergent such as TIDE® (Procter & Gamble Company, Cincinnati, OH) liquid detergent. Samples were allowed to air dry after washing.

Both the untreated and treated halves of each fabric swatch were observed visually under LED illumination prior to exposure to the anti-flea formulation, after the swatches were dry and prior to washing, and again after the samples were dry following washing. Digital photographs of each fabric swatch were obtained at each observation period to document fabric integrity and colorfastness. A secure digital high capacity (SDHC) flash memory card was used to capture the files in the camera, and was considered the source data for this Study 2.

Visual observations comparing the untreated side to the treated side of each fabric swatch did not reveal any observable changes in either fabric integrity (for example, no fraying, tearing, ripping, or other damage occurred and/or was observed) or the colorfastness for these swatches following initial exposure to the anti-flea formulation or following drying of the product on the fabric sample. Further, the visual appearance of fabric swatches following washing and drying indicated some fraying of the fabric along the edges and increased wrinkling of the samples, but these observations were similar for both the untreated side and the treated side of each fabric swatch. These observations suggested the changes in fabric integrity were likely associated with the laundering of the samples instead of the treatment with the anti-flea formulation. Still further, visual observations comparing the untreated side to the treated side of each fabric swatch did not reveal any observable changes in colorfastness for these samples following exposure to the anti-flea formulation after washing and air drying of the samples.

Based upon these observations, it is reasonable to conclude that exposure to an anti-flea formulation in accordance with the present disclosure (for example, an anti-flea formulation including spinosyn, such as an anti-flea formulation having a concentration of 2500 ppm spinosad) has no impact upon colorfastness and/or fabric integrity for the fabric type, finish and color combinations evaluated. The samples used in this study represent some of the most commonly used types of fibers and finishes used in the commercial textile industry.

Although Study 1 and Study 2 included the use of an anti-flea formulation having a 2500 ppm concentration of spinosad, it will be understood that the formulations and methods discussed herein could also include other concentrations of spinosyn (for example, spinosad), such as concentrations of between approximately 50 ppm to about 5000 ppm spinosyn.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention.

What is claimed is:

1. A method of controlling a flea infestation, the method comprising:
producing a lethal effect in all life cycle stages of fleas on an animal by topically applying a formulation to an animal, the formulation including an effective amount of at least one spinosyn and a carrier.

2. The method of claim 1, wherein in the all life cycle stages of fleas on the animal includes ova, larvae, and adults.

3. The method of claim 1, wherein the lethal effect in all life cycle stages of fleas on the animal is 100% mortality of flea ova, flea larvae, and adult fleas on the animal.

4. The method of claim 1, wherein the at least one spinosyn is spinosad.

5. The method of claim 1, wherein the formulation is an organic liquid solution.

6. The method of claim 1, wherein topically applying the formulation to the animal includes topically applying the formulation in a dosage rate of between approximately 1 mg spinosyn per kg body weight of the animal and approximately 100 mg spinosyn per kg body weight of the animal.

7. The method of claim 6, wherein topically applying the formulation to the animal includes topically applying the formulation in a dosage rate of between approximately 10 mg spinosyn per kg body weight of the animal and approximately 60 mg spinosyn per kg body weight of the animal.

8. The method of claim 6, wherein:
the formulation is a liquid formulation;
the at least one spinosyn is spinosad; and
topically applying the formulation to the animal includes topically applying between approximately 0.5 mL to approximately 6 mL of the formulation to the animal.

9. A method of controlling a flea infestation, the method comprising:
producing a lethal effect in all life cycle stages of fleas on a premises surface by applying a formulation to a premises surface, the formulation including an effective amount of at least one spinosyn and a carrier.

10. A method of claim 9, wherein the carrier is a non-organic carrier.

11. The method of claim 9, wherein the lethal effect in all life cycle stages of fleas on the premises surface is 100% mortality of flea ova, flea larvae, and adult fleas on the premises surface.

12. The method of claim 9, wherein applying the formulation to the premises surface includes at least one of spraying the formulation, soaking the premises surface in the formulation, and pouring the formulation onto the premises surface.

13. The method of claim 12, wherein applying the formulation to the premises surface includes spraying the formulation onto the premises surface to evenly cover the premises surface.

14. The method of claim 9, further comprising:
allowing the formulation to dry on the premises surface; and
then removing the dried formulation from the premises surface.

15. The method of claim 9, further comprising:
preparing the formulation before applying the formulation to the premises surface.

16. The method of claim 15, wherein the effective amount of the at least one spinosyn is present in a wettable powder and the carrier is a non-organic carrier, the preparing the formulation before applying the formulation to the premises surface including mixing the wettable powder with the non-organic carrier.

17. The method of claim 9, wherein the premises surface includes at least one of a textile, furniture upholstery, carpet, a rug, a pillow, bed linens, an animal bed, a towel, a plush toy, clothing, plastic, tile, wood, leather, concrete, glass, and metal.

18. An anti-flea formulation, the anti-flea formulation comprising:
an effective amount of a spinosyn to cause a lethal effect in all life cycle stages of flea; and
at least one carrier.

19. The anti-flea formulation of claim 18, wherein the at least one carrier is a non-organic carrier.

20. The anti-flea formulation of claim 18, wherein the spinosyn is present in an amount of between approximately 1000 ppm to approximately 4000 ppm.

21. The anti-flea formulation of claim 20, wherein the spinosyn is present in an amount of approximately 2500 ppm.

22. The anti-flea formulation of claim 18, wherein the lethal effect in all life cycle stages of flea is a 100% mortality in flea ova, flea larvae, and adult fleas.
